(19) United States
(12) Patent Application Publication
(10) Pub. No.: US 2003/0013133 A1
(43) Pub. Date: Jan. 16, 2003

(54) Detection of a biological target

(76) Inventors: Kazunori Kataoka, Tokyo (JP); Yukio Nagasaki, Moriya-shi (JP); Yuka Hori, Kashiwa-shi (JP); Masayoshi Akiyama, Nagareyama-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/182,240

(22) PCT Filed: Jan. 24, 2001
(86) PCT No.: PCT/JP01/00436

(30) Foreign Application Priority Data
Jan. 26, 2000 (JP) 2000-16843

(51) Int. Cl. G01N 33/53; G01N 33/542

(52) U.S. Cl. 435/7.9

(57) ABSTRACT

Disclosed are core-shell type polymeric micelle compositions comprising a hydrophilic-hydrophobic block polymer carrying an antigen, an antibody or the like on the hydrophilic region wherein a compound undergoing a change in physical characteristic in response to a structural change of the micelles or a part thereof has been incorporated into the core region. These compositions can be used in an indirect agglutination reaction (or a passive agglutination reaction) for assaying an analyte (an antibody, an antigen or the like) in a biological sample.

### TECHNICAL FIELD

This invention relates to compositions for the detection of an analyte present in a biological sample, and a method for the detection of such an analyte.

### BACKGROUND ART

In the field, for example, of serodiagnosis, there are assay methods utilizing an indirect agglutination reaction (or passive agglutination reaction). In these methods, a specific antibody or a specific antigen is generally detected by using bentonite, polystyrene latex, erythrocytes, bacterial cells or the like as a carrier for the antibody or antigen. Among them, polystyrene latex is widely used for indirect agglutination reactions because it can be easily formed into particles having a uniform size and the particles themselves have no antigenicity. This polystyrene latex is highly suitable for the purpose of immobilizing an antigen or antibody, in that it adsorbs proteins and the like strongly. Conversely, it is likely to adsorb various components present in the reaction system nonspecifically and thereby produce an erroneous result. Moreover, in order to achieve an accurate detection by utilizing this system, a considerable length of time is required for a latex agglutination reaction induced by the formation of an immune complex from an antibody or antigen present in a sample and a corresponding antigen or antibody present on latex particles. Furthermore, in this system where aggregates of particles are to be detected, a high background level may result depending on the assay method.

Accordingly, there still remains a need for a detection means which, in the detection of an analyte present in a biological sample, can be utilized for various assay system including the above-described indirect agglutination reaction system and in which the disadvantages associated with the use of the aforesaid polystyrene latex are eliminated or overcome.

### DISCLOSURE OF THE INVENTION

The present inventors have conducted investigations as to the provision of various hydrophilic-hydrophobic block polymers and the characterization and use thereof. According to an investigation on the stability of polymeric micelles formed by those block polymer which was conducted as a part of such research, it has been found that a structural change of individual polymeric micelles associated with the agglutination of the polymeric micelles under certain conditions (leading eventually to the disintegration of the micelles) can be rapidly and sensitively traced by a specific compound disposed in the polymeric micelles.

In further amplification, it may be said that, when a plurality of polymeric micelles agglutinate or combine specifically through the medium of an analyte present in a biological sample, the presence of the analyte can be indirectly detected through a structural change of the polymeric micelles. The present invention has been completed on the basis of the above-described findings.

Thus, according to the present invention, there is provided a composition comprising a hydrophilic-hydrophobic block polymer for detecting an analyte present in a biological sample, wherein:

the analyte is capable of becoming one of the constituent members forming a specifically binding pair by non-covalent bonding; and

the block polymer forms core-shell type polymeric micelles, the other of the constituent members capable of forming the specifically binding pair with the analyte is covalently bonded to the hydrophilic block forming the shell of the polymeric micelles, and a compound undergoing a change in physical characteristic in response to a structural change of the polymeric micelles is disposed in the core part of the polymeric micelles.

According to another embodiment of the present invention, there is provided a method for detecting an analyte present in an aqueous solution, the method comprising the steps of

(A) mixing an aqueous solution suspected of containing the analyte, with core-shell type polymeric micelles formed from a hydrophilic-hydrophobic block polymer in which a member capable of forming a specifically binding pair with the analyte is covalently bonded to the hydrophilic block and a compound undergoing a change in physical characteristic in response to a structural change of the micelles is disposed in the core part;

(B) incubating the mixture obtained in step (A) to effect a structural change of the polymeric micelles through the formation of a complex between the analyte and the member capable of forming a specifically binding pair therewith in the polymeric micelles; and

(C) relating a change in physical characteristic caused by the structural change of the polymeric micelles, to the amount of analyte present.

### BRIEF DESCRIPTION OF THE DRAWINGS

FIG. 1 shows fluorescence spectra recorded (a) before and (b) after the contact of polymeric micelles (biotin-PEG-PLA micelles) with avidin.

### BEST MODE FOR CARRYING OUT THE INVENTION

The hydrophilic-hydrophobic block polymer used in the present invention may be any hydrophilic-hydrophobic block polymer, provided that it forms polymeric micelles in an aqueous medium and meets the purposes of the present invention. Suitable aqueous media include purified water, a buffered aqueous solution, an aqueous solution containing an inorganic salt, an aqueous solution containing a water-miscible organic solvent such as ethanol, acetone or dimethylformamide, and the like. The term “polymeric micelle” as used herein refers to a so-called copolymer micelle and means a molecular aggregate produced by the association of polymer molecules so as to form a core (consisting chiefly of hydrophobic segments)—shell (consisting chiefly of hydrophilic segments) structure in an aqueous medium. Moreover, the expression “block polymer which meets the purposes of the present invention” means a block polymer capable of forming polymeric micelles which at least individually exist stably in an aqueous medium, but when two or more polymeric micelles agglutinate indirectly through the medium of an analyte, undergo a structural change to form a single aggregate of polymeric micelles at the minimum.

Such a hydrophilic-hydrophobic block polymer may be selected from among those well known in the art. Among others, those which have been investigated for use as carriers for drugs hardly soluble in water. Specifically, this block polymer has a structure containing a linear hydrophilic polymer block (or segment) and a linear hydrophobic polymer block (or segment).

Preferred examples of the hydrophilic polymer block include, but are not limited to, ones consisting essentially of uncharged segments of polyethylene oxide or polyethylene glycol, polyvinyl alcohol, polyvinyl pyrrolidone and the like. Among them, a hydrophilic polymer block consisting entirely of a polyethylene oxide segment is especially preferred. On the other hand, preferred examples of the hydrophobic polymer block include ones consisting essentially of poly(lactide), poly(ε-caprolactone), poly(δ-valerolactone), poly(γ-butyrolactone), poly(β-benzyl aspartate), poly(γ-benzyl glutamate), poly(valine), poly(leucine), poly(methacrylate)s, poly(acrylate)s and the like. Among them, a hydrophobic polymer block consisting entirely of a poly(lactide) segment is preferred, though the present invention is not limited thereto. The expression “consisting essentially of” means that the indicated polymer segments comprise at least 95% of each block. The block copolymers containing any combination of such hydrophilic blocks and hydrophobic blocks are hydrophilic-hydrophobic block copolymers in accordance with the present invention, provided that they meet the purposes of the present invention. It is to be understood that the term “poly” used herein to show each segment of the block polymer also comprehends the meaning of “oligo”, so long as a block polymer meeting the purposes of the present invention can be formed.

As the above-defined block polymer, any of the block polymers described, for example, in WO93/16687, U.S. Pat. No. 5,410,016, Japanese Patent Laid-Open No. 107565/'94, WO96/33233, WO96/32434 and WO97/06202 may be used directly or with further modifications. Among others, it is especially preferable to select a block polymer as described in the aforementioned WO96/33233, WO96/32434 and WO97/06202, which is represented by the general formula

A—(CH₂CH2O)ₙ—(Y)ₘ—Z

wherein A is a residue having any of various functional groups, Y is

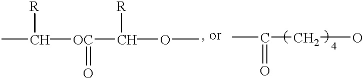

Z is a residue having any of various functional groups, R is an alkyl group, and m and n are independently integers of 5 to 2,000, provided that the values of m and n are not limited thereto but are chosen so as to enable the block polymer to form polymeric micelles in an aqueous medium, and use it with a suitable modification. Such a modification may be made, for example, through the medium of a functional group which can be present in the aforesaid A or Z moiety, such as an aldehyde (—CHO), amino (—NH₂), mercapto (—SH), hydroxyl (—OH), carboxyl (—COOH) or vinyl (—CH═CH₂) group.

According to the present invention, such a block polymer is used in the form of core-shell type polymeric micelles in which a constituent member (other than an analyte) capable of forming a specifically binding pair with the analyte is covalently bonded to the hydrophilic block forming the shell. Thus, one member of the specifically binding pair is present in the shell region of the polymeric micelles or on the surface thereof.

The specifically binding pair may be any pair that consists of at least two constituent members capable of forming a biochemical complex, conjugate or the like by hydrogen bonding, hydrophobic bonding or other non-covalent bonding, provided that the constituent members are specifically combined. Specific examples of such pairs include, but are not limited to, combinations of an antigen and an antibody; biotin and avidin; a sugar and lectin; a hormone or signal transfer substance and a corresponding receptor; an enzyme and a substrate or inhibitor therefor; a (DNA or RNA) fragment comprising a certain nucleotide sequence and a DNA or RNA fragment forming a hybrid with the sequence under stringent conditions (see, for example, Hamers and Higgins ed., Nucleic Acid Hybridisation, IRL Press, Oxford, U.K.); and the like. Accordingly, any one of the members forming the aforesaid pair is covalently bonded to the block polymer used in the present invention, through the medium of a functional group present in the hydrophilic block thereof and, in particular, the A moiety in the above general formula. This covalent bond may be formed according to a per se known method utilizing, for example, condensation, addition substitution and, if necessary, an oxidation or reduction reaction.

Since the “analyte present in a biological sample” as used herein is one of the constituent members capable of forming a specifically binding pair as described above, it can be any one member constituting one of the combinations enumerated above as specific examples of the pair. For example, it can be an antigen or an antibody, a nucleic acid or a fragment thereof, or the like. The biological sample may be any natural or artificial sample that has the possibility of containing the aforesaid analyte. Natural samples include blood, urine, sweat, saliva, diluted or concentrated preparations thereof, and the like. Artificial samples include cultures of animal, plant or microbial cells, disintegrated products of these cells, synthesized mixtures of peptides or nucleic acids, and the like. If necessary, these biological samples may be in the form of aqueous solutions buffered with an appropriate buffering agent.

In the core-shall type polymeric micelles used in the present invention, a compound undergoing a change in physical characteristic in response to a structural change of the micelles is disposed in the core part (or region). The physical characteristic can be light absorption or emission intensity or other type of energy that is influenced by changes of the environment surrounding the compound, such as a change of the electronic state of the molecule, a structural change and a change in interaction with the medium. Typical examples of such compounds undergoing a change in physical characteristic include fluorescent compounds, and polycyclic aromatic compounds (e.g., pyrene and its derivatives) characterized in that two or more adjacent molecules can form an excimer. Fluorescent compounds are commonly used in the field of immunoassay. In particular, there may be used any fluorescent compounds having high fat solubility. Such a compound can generally be disposed in the core part of the polymeric micelles, by allowing it to coexist in the solution being treated to form polymeric micelles from the above-described block polymer. Although the present inventors do not wish to be bound by any theory, it is believed that, in the core part of the polymeric micelles, the aforesaid compound is present in a concentrated or mutually adjoining state. When two or more polymeric micelles agglutinate through the medium of an analyte, the individual polymeric micelles undergo a structural change (leading eventually to the disintegration of the micelles). As a result, the aforesaid compound is dispersed or released to cause a change in fluorescence intensity or a change such as the disappearance of excimer emission.

The term “physical characteristic” as used herein also comprehends electron spin resonance (ESR) absorption intensity, ¹³C nuclear magnetic resonance (NMR) absorption intensity and the like. Substances exhibiting ESR absorption generally include substances which can be used as probes in the spin labeling method. Specifically, they include substances forming stable nitroxide radicals, such as 2,2,6,6-tetramethylpiperidine N-oxide derivatives and 4,4-dimethyloxazolidine N-oxide derivatives. Normally, when these probes are present in the core of polymeric micelles, little ESR signal is generated. However, the disintegration of micelles causes a signal to be observed. On the other hand, NMR probes (e.g., ¹³C-enriched acetyl group) exhibit the same absorption behavior as ESR probes.

According to the present invention, such a change usually occurs rapidly at room temperature (18-27° C.) and is so marked as to give a very high sensitivity.

In a preferred embodiment, a compound undergoing a change in physical characteristic in response to a structural change of the polymeric micelles is covalently bonded to the hydrophobic segment of the block polymer, for example, through the medium of a functional group present in the Z moiety of the above general formula. Thereafter, polymeric micelles are formed from this block polymer. Thus, the part arising from the compound can be disposed in the state covalently bonded to the core part of the micelles. This covalent bond may be formed by a per se known reaction in which the aforesaid compound having a functional group capable of being covalently bonded to the functional present in the Z moiety or, if necessary, a compound having such a functional group introduced thereinto is bonded through the medium of the functional group present in the Z moiety.

The above-described polymeric micelles may be formed according to any of various per se known methods including, for example, the methods described in the aforementioned WO96/33233, WO96/33234 and WO97/06202. The polymeric micelles so formed may be used as a composition in accordance with the present invention, if necessary, after it is prepared in the form of a desalted, desolvated or buffered aqueous solution. This composition generally comprises micelles of nanometer size and is hence transparent, resulting in a reduction in background level.

The present invention also provides a method for detecting an analyte present in a biological sample by using the above-described composition. According to this method, the aforesaid composition is mixed with a biological sample suspected of containing an analyte, and the resulting mixture is incubated. This mixing may be carried out in a cuvette attached to an analyzer having the function of measuring light absorption or fluorescence intensity, or in a well of a microtiter plate. For this purpose, any of various apparatus and devices commonly used in this technical field may be used in an ordinary fashion without any modification.

If the analyte which can combine with a constituent member present in the shall part of the polymeric micelles and capable of forming a specifically binding pair is present in the sample, two or more polymeric micelles undergo an indirect agglutination reaction through the medium of the analyte. As a result, the individual micelles undergo a structural change. In response to this structural change, the compound disposed in the core part of the polymeric micelles (e.g., a fluorescent compound, a polycyclic aromatic compound such as pyrene, an ESR probe, or an NMR probe) exhibits a change in physical property, such as a rise in fluorescence intensity, the disappearance of excimer emission, the generation of an ESR signal, or the generation of a signal arising from ¹³C. Then, this change can be measured and the measured value can be related to the amount of analyte present in the sample.

Thus, according to the method of the present invention for the detection of an analyte, an antigen or an antibody present in a biological sample, or any one of the constituent members capable of forming another biologically specific complex or conjugate can be detected rapidly and sensitively.

The present invention is further illustrated by the following specific examples. However, it is to be understood that the present invention is not limited thereto.

### EXAMPLE 1

### Preparation of a Block Polymer

(1) Preparation of acetal-PEG/PLA-OH

20 ml of tetrahydrofuran (THF) was placed in a reaction vessel under an atmosphere of argon. Then, using 3,3-diethoxy-1-propanol (0.1 mmol) as a starting material and potassium naphthalene (0.1 mmol) as a metallizing agent, metallization was carried out for 10 minutes. Ethylene oxide (114 mmol) was added to this solution, followed by stirring at room temperature for 2 days. Then, lactide (35 mmol) was added thereto, followed by stirring at room temperature for 3 hours. Thereafter, the reaction vessel was unsealed and the reaction was stopped. The polymer was purified by pouring the reaction mixture into cold isopropanol to precipitate the polymer (acetal-PEG/PLA-OH) and further reprecipitating it with cold isopropanol. After the solvent was distilled off, the resulting residue was dissolved in benzene and freeze-dried to recover the polymer. Its yield after purification was about 85%. The molecular weights of the polyethylene segment (PEG) and the polylactide segment (PLA) were 5,000 and 5,500, respectively.

(2) Preparation of acetal-PEG/PLA-Py (pyrene)

To a reaction vessel containing 100 ml of acetonitrile, acetal-PEG/PLA-OH (0.03 mmol), quinuclidine (about 50 parts based on the polymer) and pyrene-1-carbonyl cyanide (about 17 parts based on the polymer) were added, followed by stirring at 60° C. for 1 hour. Thereafter, the reaction mixture was dialyzed against dimethyl sulfoxide (DMSO) for 4 days (by using a dialysis membrane having a fractionating molecular weight of 1,000 and replacing DMSO once a day) and then dialyzed against water for 1 day (by using a dialysis membrane having a fractionating molecular weight of 1,200 to 14,000 and replacing distilled water twice). After the solvent was removed by freeze-drying, the resulting residue was dissolved in THF and reprecipitated with ether. The precipitate was collected by suction filtration, dissolved in benzene, and freeze-dried to recover the title polymer. Its yield was about 70%.

(3) Preparation of acetal-PEG/PIA-Py micelles

The acetal-PEG/PLA-Py was dissolved in 30 ml of dimethylacetamide (DMAc), and this solution was dialyzed against water for 1 time (by using a dialysis membrane having a fractionating molecular weight of 12,000 to 14,000 and replacing distilled water after 2, 5 and 8 hours).

(4) Preparation of biotin-PEG/PLA-Py micelles

The aforesaid acetal-PEG/PLA-Py micelle solution was adjusted to pH 2 with hydrochloric acid and subjected to a deprotection reaction for 2.5 hours. Thereafter, the solution was adjusted to pH 5 with sodium hydroxide and subjected to desalting dialysis for 1 day (by using a dialysis membrane having a fractionating molecular weight of 12,000 to 14,000 and replacing distilled water after 2 hours). After the concentration of the recovered aldehyde-type micelle solution was adjusted to 2 mg/ml, biotin-hydrazide (1.5 parts based on the polymer) was added thereto. The resulting mixture was reacted at 50° C. for 5 hours and dialyzed against water at 50° C. for 3 days (by using a dialysis membrane having a fractionating molecular weight of 1,000 and replacing distilled water once a day). Thereafter, the resulting biotin-PEG/PLA-Py block copolymer was recovered.

### EXAMPLE 2

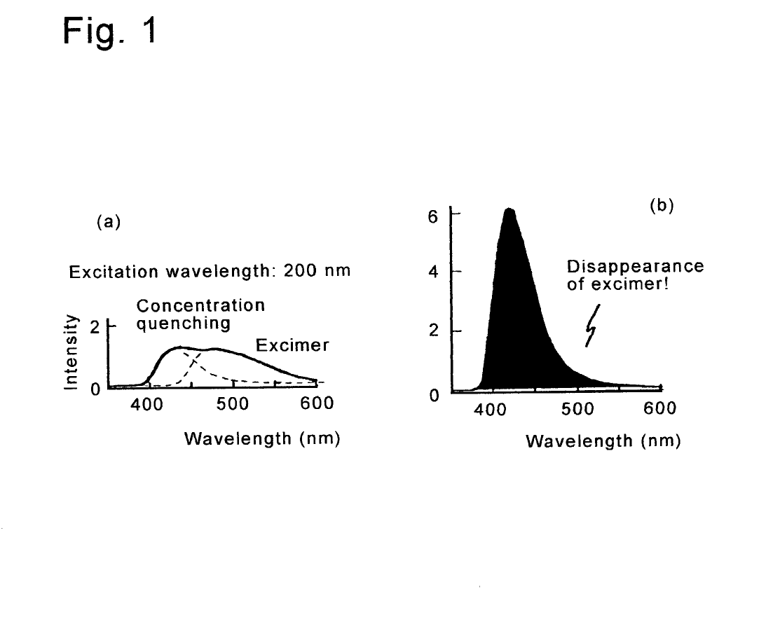

100 mg of the biotin-PEG-PLA-Py block polymer was dissolved in 20 ml of DMAc, and this solution was dialyzed against 2 L of water for 24 hours (by replacing water after 3, 6 and 9 hours) (MWCO=12 K to 14K). The resulting core-shell type PEG-PLA micelles having biotin on the surface and pyrene in the core were adjusted to a concentration of 0.1 mg/ml with a phosphate buffer solution (having a pH of 7.2 and an ionic strength of 0.2 M). To 3 ml of this solution was added a PBS solution containing the same site amount of avidin. When the fluorescence of this mixture was measured, the fluorescence intensity at 420 nm rose from 1.2 to 6. Moreover, the excimer emission at 500 nm disappeared. The fluorescence spectra recorded before and after the addition of avidin are shown in FIG. 1.

### EXAMPLE 3

A biotin-PEG-PLA-Py block polymer micelle solution was prepared in the same manner as in Example 2, and 30 μl of anti-biotin antibody [50 mM in PBS (pH 7.4)] was added to 3 ml of the solution. When the fluorescence of this mixture was measured, the fluorescence intensity at 420 nm rose from 1.2 to 6.5. Moreover, the excimer emission at 500 nm disappeared.

### EXAMPLE 4

A micelle solution was prepared in the same manner as in Example 2, except that galactose-PEG-PLA-Py was used. To 3 ml of this solution was added a PBS solution containing a tenfold molar amount of lectin protein (castor beans). When the fluorescence of this mixture was measured, the fluorescence intensity at 420 nm rose from 1.2 to 6.3. Moreover, the excimer emission at 500 nm disappeared.

### INDUSTRIAL APPLICABILITY

The present invention provides compositions permitting an analyte present in a sample to be sensitively detected, the analyte being any one member of a specifically binding pair capable of forming a biochemical complex or conjugate (e.g., an antibody or an antigen, or a polynucleotide or an oligonucleotide having a sequence complementary thereto), as well as a method for the detection of such an analyte. Accordingly, the present invention can be utilized, for example, in the industry for the manufacture of diagnostic drugs or test drugs.

1. A composition comprising a hydrophilic-hydrophobic block polymer for detecting an analyte present in a biological sample, wherein:
the analyte is capable of becoming one of the constituent members forming a specifically binding pair by non-covalent bonding; and
the block polymer forms core-shell type polymeric micelles, the other of the constituent members capable of forming the specifically binding pair with the analyte is covalently bonded to the hydrophilic block forming the shell of the polymeric micelles, and a compound undergoing a change in physical characteristic in response to a structural change of the polymeric micelles is disposed in the core part of the polymeric micelles.

2. A composition as claimed in claim 1 wherein the hydrophilic-hydrophobic block polymer has a hydrophilic block comprising a polyethylene segment.

3. A composition as claimed in claim 1 wherein the hydrophilic-hydrophobic block polymer has a hydrophobic block comprising a polylactide segment.

4. A composition as claimed in claim 1 wherein the hydrophilic-hydrophobic block polymer contains a polyethylene segment and a polylactide segment.

5. A composition as claimed in claim 1 wherein the compound undergoing a change in physical characteristic in response to a structural change of the polymeric micelles is covalently bonded to the hydrophobic block of the hydrophilic-hydrophobic block polymer.

6. A composition as claimed in claim 1 wherein the physical characteristic changing in response to a structural change of the polymeric micelles is selected from fluorescence intensity, electron spin resonance absorption intensity and ¹³C nuclear magnetic resonance absorption intensity.

7. A composition as claimed in claim 6 wherein the physical characteristic changing in response to a structural change of the polymeric micelles is fluorescence intensity.

8. A composition as claimed in claim 1 wherein the specifically binding pair is selected from the group consisting of a pair of an antigen and an antibody, a pair of biotin and avidin, a pair of a sugar and lectin, a pair of a hormone or signal transfer substance and a corresponding receptor protein, a pair of an enzyme and a substrate or inhibitor therefor, and a pair of a nucleic acid fragment comprising a specific nucleotide sequence, and a nucleic acid fragment or oligonucleotide capable of forming a hybrid with the sequence under stringent conditions.

9. A composition as claimed in claim 1 wherein the other of the constituent members capable of forming the specifically binding pair, which is covalently bonded to the hydrophilic block, is an antibody or an antigen, or a polynucleotide or oligonucleotide.

10. A method for detecting an analyte present in a biological sample, the method comprising the steps of
(A) mixing a biological sample suspected of containing the analyte, with core-shell type polymeric micelles formed from a hydrophilic-hydrophobic block polymer in which a member capable of forming a specifically binding pair with the analyte is covalently bonded to the hydrophilic block and a compound undergoing a change in physical characteristic in response to a structural change of the micelles is disposed in the core part;
(B) incubating the mixture obtained in step (A) to effect a structural change of the polymeric micelles through the formation of a complex between the analyte and the member capable of forming a specifically binding pair therewith in the polymeric micelles; and
(C) relating a change in physical characteristic caused by structural change of the polymeric micelles, to the amount of analyte present.

11. A method as claimed in claim 10 wherein the hydrophilic-hydrophobic block polymer contains a polyethylene segment as the hydrophilic block and a polylactide segment as the hydrophobic block.

12. A method as claimed in claim 11 wherein the physical characteristic changing in response to a structural change of the polymeric micelles is selected from fluorescence intensity, electron spin resonance absorption intensity and ¹³C nuclear magnetic resonance absorption intensity.
